(19) United States
(12) Patent Application Publication
(10) Pub. No.: US 2005/0256163 A1
(43) Pub. Date: Nov. 17, 2005

(54) Crystalline forms of fexofenadine hydrochloride and processes for their preparation

(76) Inventors: Ilan Kor, Shoham (IL); Shlomit Wizel, Petah Tiqva (IL)

Correspondence Address: KENYON & KENYON, ONE BROADWAY, NEW YORK, NY, 10004, US

(21) Appl. No.: 11/115,808

(22) Filed: Apr. 26, 2005

Related U.S. Application Data
(60) Provisional application No. 60/565,559, filed on Apr. 26, 2004.
(60) Provisional application No. 60/581,877, filed on Jun. 21, 2004.
(60) Provisional application No. 60/583,233, filed on Jun. 25, 2004.

(51) Int. Cl. C07D 211/20; A61K 31/445

(52) U.S. Cl. 514/317; 546/237

(57) ABSTRACT

Provided are crystalline forms of fexofenadine hydrochloride and processes for their preparation.

### FIELD OF THE INVENTION

The present invention relates to the solid state chemistry of fexofenadine hydrochloride.

### BACKGROUND OF THE INVENTION

4-[4-[4-(hydroxydiphenylmethyl)-1-piperidinyl]-1-hydroxybutyl]-α,α-dimethylbenzeneacetic acid of Formula (I) (fexofenadine) is an H₁ receptor antagonist and a useful antihistaminic drug. It has low permeability into central nervous system tissues and weak antimuscarinic activity, causing it to have few systemic side effects. 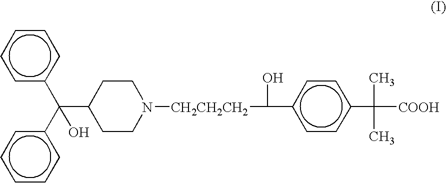
It has low permeability into central nervous system tissues and weak antimuscarinic activity, causing it to have few systemic side effects.

The antihistamic activity of fexofenadine is disclosed in U.S. Pat. No. 4,254,129, incorporated herein by reference. According to the '129 patent, fexofenadine can be prepared starting from ethyl, α,α-dimethylphenyl acetate and 4-chlorobutyroyl chloride, which are reacted under Freidel-Crafts conditions. Chloride is displaced from the Freidel-Crafts product with α,α-diphenyl-4-piperidine-methanol to give 4-[4-[4-(hydroxydiphenylmethyl)-1-piperidinyl]-1-oxobuty-1]-α,α-dimethylbenzeneacetate, which is isolated as its hydrochloride salt. The ketone is then reduced with PtO/H₂ and the ester group is hydrolyzed to yield fexofenadine hydrochloride.

Other methods of preparing fexofenadine are discussed in U.S. Pat. Nos. 5,578,610, 5,589,487, 5,581,011, 5,663,412, 5,750,703, 5,994,549, 5,618,940, 5,631375, 5,644,061, 5,650,516, 5,652,370, 5,654,433, 5,663,353, 5,675,009, 5,375,693 and 6,147,216.

The present invention relates to the solid state physical properties, i.e., polymorphism, of fexofenadine hydrochloride. These properties may be influenced by controlling the conditions under which fexofenadine hydrochloride is obtained in solid Form. Solid state physical properties include, for example, the flowability of the milled solid. Flowability affects the ease with which the material is handled during processing into a pharmaceutical product. When particles of the powdered compound do not flow past each other easily, a Formulation specialist must take that fact into account when developing a tablet or capsule Formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences because it imposes an upper limit on the rate at which an orally-administered active ingredient may reach the bloodstream. The rate of dissolution is also a consideration in Formulating syrups, elixirs and other liquid medicaments. The solid state Form of a compound may also affect its behavior on compaction and its storage stability.

These practical physical characteristics are influenced by the conformation and orientation of molecules in the unit cell, which defines a particular polymorphic Form of a substance. The polymorphic Form may give rise to thermal behavior different from that of the amorphous material or another polymorphic Form. Thermal behavior is measured in the laboratory by such techniques as capillary melting point, thermogravimetric analysis (TGA) and differential scanning calorimetry (DSC), and may be used to distinguish some polymorphic Forms from others. A particular polymorphic Form may also give rise to distinct properties that may be detectable by powder X-ray diffraction, solid state 13C NMR spectrometry and infrared spectrometry.

U.S. Pat. Nos. 5,738,872, 5,932,247 and 5,855,912, incorporated herein by reference, describe four crystal Forms of fexofenadine hydrochloride which are designated Forms I-IV. According to the '872 and related patents, Forms II and IV are hydrates and Forms I and III are anhydrates. Each Form is characterized by its melting point, onset of endotherm in the DSC profile, and PXRD.

The '872 patent discusses methods of interconverting Forms I-IV. Aqueous recrystallization of Form I can be used to produce Form II. Water-minimizing recrystallization or azeotropic distillation of either Form II or Form IV can yield Form I. Form III is reported to be accessible by water minimizing recrystallization of Form II. Crystal digestion of Form III can be used to obtain Form I. Forms II and IV can be obtained directly by sodium borohydride reduction of 4-[4-[4-(hydroxydiphenylmethyl)-1-piperidinyl]-1-oxobutyl]-alpha, alpha-.-dimethylbenzeneacetate as described in Examples 1 and 2.

Fexofenadine hydrochloride Forms V, VI, and VIII through XV are disclosed in US 20030021849 and US 20020177608 (WO02/080857), both of which are incorporated herein by reference.

Fexofenadine hydrochloride Form XVI is disclosed in US 20040044038, in which fexofenadine hydrochloride Form XVI is characterized by a powder XRD pattern with peaks at 10.1, 15.2, 18.6, 19.2, 20.1±0.2 degrees two theta. According to the publication, Form XVI has a DSC profile with two endothermic peaks at a temperature range of up to about 125° C. and an additional endotherm at a temperature of about 135° C. Form XVI also has a TGA thermogram with a loss on drying (LOD) of about 6% to about 10% at a temperature range of up to about 145° C.

There is a need in the art for additional polymorphic forms of fexofenadine hydrochloride and processes on industrial scale for their preparation.

### SUMMARY OF THE INVENTION

In one aspect, the present invention provides a crystalline form of fexofenadine HCl (Form XIX) characterized by a powder XRD pattern with peaks at: 3.8, 8.8, 11.3, 18.8, 20.2±0.2 deg. 2θ. The crystalline form may further be characterized with a DSC profile having a first endothermic peak at a temperature of about 90° C. to about 100° C. and a second endotherm at a temperature of about 148 to about 155° C.; or a weight loss of about 4 to about 8% at temperature range of 30° C. to 150° C. by TGA. Also provided is a powder of the crystalline form comprising less than 5% by weight of any other crystalline form of fexofenadine hydrochloride.

In one aspect, the present invention provides a process for preparing the above crystalline fexofenadine hydrochloride comprising:
a) preparing a solution of fexofenadine hydrochloride in C₁-C₄ alcohol having at least about 15% water by volume relative to the C₁-C₄ alcohol, wherein ratio of fexofenadine base used to prepare the fexofenadine hydrochloride to the C₁-C₄ alcohol is about 1:2.5 to about 1:4 (g/vol);
b) cooling the solution to crystallize the crystalline form; and
c) recovering the crystalline form.
   Preferably cooling is carried out to a temperature of about 0° C. to about 10° C. Preferably the C₁-C₄ alcohol is methanol. Preferably prior to the recovering step an anti-solvent is added to the solution. Preferably the anti-solvent is a C₅ to C₁₂ saturated hydrocarbon. Preferably the crystalline fexofenadine hydrochloride recovered has less than about 5% by weight of any other crystalline form of fexofenadine hydrochloride. Preferably other crystalline forms are present in less than about 2% by weight.

In another aspect the present invention provides a process for preparing crystalline fexofenadine hydrochloride (Form XVI) with peaks at 10.1, 15.2, 18.6, 19.2, 20.1±0.2 degrees two theta comprising:
a) preparing a solution of fexofenadine hydrochloride in a mixture of water and C₁-C₄ alcohol having less than about 12% water by volume relative to the alcohol;
b) cooling the solution to crystallize the crystalline form; and
c) recovering the crystalline form.
   Preferably the C₁-C₄ alcohol is selected from the group consisting of methanol and isopropyl alcohol. Preferably the amount of water is of about 5% to about 12% by volume. Preferably the amount of water is about 10%. Preferably the solution is cooled to a temperature of less than about negative 5° C. Preferably the solution is cooled to a temperature of less than about negative 12° C. Preferably prior to recovering the crystalline form an anti-solvent is added to the solution. Preferably the anti-solvent is a C₅ to C₁₂ saturated hydrocarbon. Preferably the crystalline fexofenadine hydrochloride recovered has less than about 5% by weight of any other crystalline form of fexofenadine hydrochloride. Preferably any other crystalline forms are present in less than about 2% by weight.

In another aspect the present invention provides a crystalline Form of fexofenadine HCl (Form XXI) characterized by a powder XRD pattern with peaks at 7.2, 11.7, 14.1, 15.4, 16.9, 18.5, 23.1, and 23.9±0.2 deg. 2θ. Also provided is a powder of the crystalline form comprising less than 5% by weight another crystalline form fexofenadine hydrochloride.

In another aspect the present invention provides process for preparing crystalline fexofenadine hydrochloride Form XXI comprising:
a) preparing a solution of fexofenadine HCl in isopropanol having at least about 10% water by volume, wherein the ratio of fexofenadine base used to prepare the fexofenadine hydrochloride to isopropanol is no more than about 1:2 (g/vol);
b) cooling the solution to crystallize the crystalline form; and
c) recovering the crystalline form.

Preferably the solution is cooled to a temperature of about −20° C. to about 0° C. Preferably the solution is cooled to a temperature of about −10° C.

In another aspect the present invention for a crystalline form of Fexofenadine HCl (Form XX) characterized by a powder XRD pattern with peaks at 5.4, 10.7, 14.0, 14.7, 15.8, 17.0, 19.0, 20.0, 21.6 and 23.2±0.2 deg. 2θ. The crystalline form may also be characterized by a DSC profile with a first endothermic peak at a temperature of about 50-55° C. and a second endotherm at a temperature of about 100° C. and about 140° C. Also provided is a powder of crystalline form of the crystalline form comprising less than 5% by weight another crystalline form of fexofenadine hydrochloride.

In another aspect the present invention provides a process for preparing crystalline fexofenadine hydrochloride Form XX comprising drying for a sufficient time a crystalline fexofenadine hydrochloride (Form XVI) having a powder XRD pattern with peaks at 10.1, 15.2, 18.6, 19.2, 20.1±0.2. In one embodiment, the drying is carried out for at least about 10 hours.

In one embodiment the drying is carried out with one of
a) a tray dryer;
b) mixed vacuum bed drier.
   In one embodiment the tray dryer is a tray vacuum dryer. In one embodiment the drying is carried out at a temperature of about 75° C. to 90° C. In one embodiment the mixed vacuum drying is carried out at a temperature of about 60° C. to about 70° C. In one embodiment the drying with the fluidized bed drier carried out at a temperature of about 20° C. to about 30° C. In one embodiment the fexofenadine hydrochloride is vigorously mixed during drying. In one embodiment the fexofenadine hydrochloride is seeded during or after drying.

In another aspect, the present invention provides a process for preparing crystalline fexofenadine hydrochloride XX comprising micronizing fexofenadine hydrochloride Form XVI with a micronizer. Preferably feed air pressure into the micronizer is of about 6 to about 8 bar. Preferably the grinding air pressure of the micronizer is of about 4 to about 7 bar.

In another aspect, the present invention provides a process for converting crystalline fexofenadine hydrochloride Form XX to crystalline fexofenadine HCl (Form XVI) with peaks at 10.1, 15.2, 18.6, 19.2, 20.1±0.2 comprising exposing Fexofenadine HCl Form XX to a relative humidity of greater than about 40%. Preferably the relative humidity is about 70% to about 85%. Preferably fluidized bed or controlled humidity cells are used. Preferably the crystalline form is obtained with at least 80% yield. Preferably the temperatures is below about 35° C. Preferably the temperatures is about room temperature.

In another aspect, the present invention provides a process for preparing fexofenadine HCl amorphous comprising heating crystalline fexofenadine hydrochloride (Form XVI) with peaks at 10.1, 15.2, 18.6, 19.2, 20.1±0.2. Preferably the temperature is about 80° C. to about 100° C.

In another aspect, the present invention provides a pharmaceutical composition comprising a crystalline fexofenadine hydrochloride selected from the group consisting of Form XIX, XX, XXI and mixtures thereof, and a pharmaceutically acceptable excipient.

In another aspect, the present invention provides a pharmaceutical composition comprising a crystalline fexofenadine hydrochloride selected from the group consisting of Form XIX, XX, XXI and mixtures thereof, and a pharmaceutically acceptable excipient, for use in reducing serotonin re-uptake in a mammal in need thereof.

In another aspect, the present invention provides a method of reducing serotonin re-uptake in a mammal comprising administering the pharmaceutical composition of the present invention to the mammal in need thereof.

### BRIEF DESCRIPTION OF THE FIGURES

FIG. 1 is a powder XRD pattern for fexofenadine hydrochloride Form XVI.

FIG. 2 is DSC thermogram for fexofenadine hydrochloride Form XVI.

FIG. 3 is a TGA thermogram for fexofenadine hydrochloride Form XVI.

FIG. 4 is a powder XRD pattern for fexofenadine hydrochloride Form XIX.

FIG. 5 is DSC thermogram for fexofenadine hydrochloride Form XIX.

FIG. 6 is a TGA thermogram for fexofenadine hydrochloride Form XIX.

FIG. 7 is a powder XRD pattern for fexofenadine hydrochloride Form XX.

FIG. 8 is DSC thermogram for fexofenadine hydrochloride Form XX.

FIG. 9 is a TGA thermogram for fexofenadine hydrochloride Form XX.

FIG. 10 is a powder XRD pattern for fexofenadine hydrochloride Form XXI.

FIG. 11 is a powder XRD pattern of Fexofenadine HCl Form XVI prepared according to Example 2.

FIG. 12 is a powder XRD pattern of a sample containing a mixture of Fexofenadine HCl Form XVI and Fexofenadine HCl Form XX prepared by exposing Fexofenadine HCl Form XVI to 0% RH atmosphere according to Example 4.

FIG. 13 is a powder XRD pattern of a sample containing a mixture of Fexofenadine HCl Form XVI and amorphous Form, prepared by heating Fexofenadine HCl Form XVI to 100° C. for 10 hours (Example 6).

FIG. 14 is a microscope observation of fexofenadine HCl Form I (top) and fexofenadine HCl form XX (bottom).

FIG. 15 is a comparison of solubility of fexofenadine HCl Form I and fexofenadine HCl form XX.

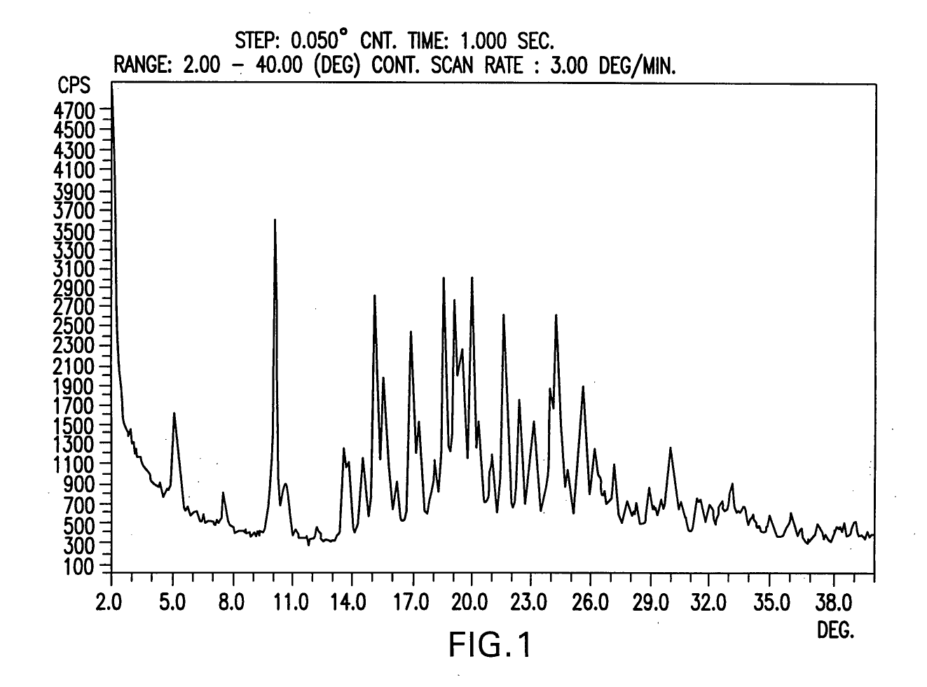

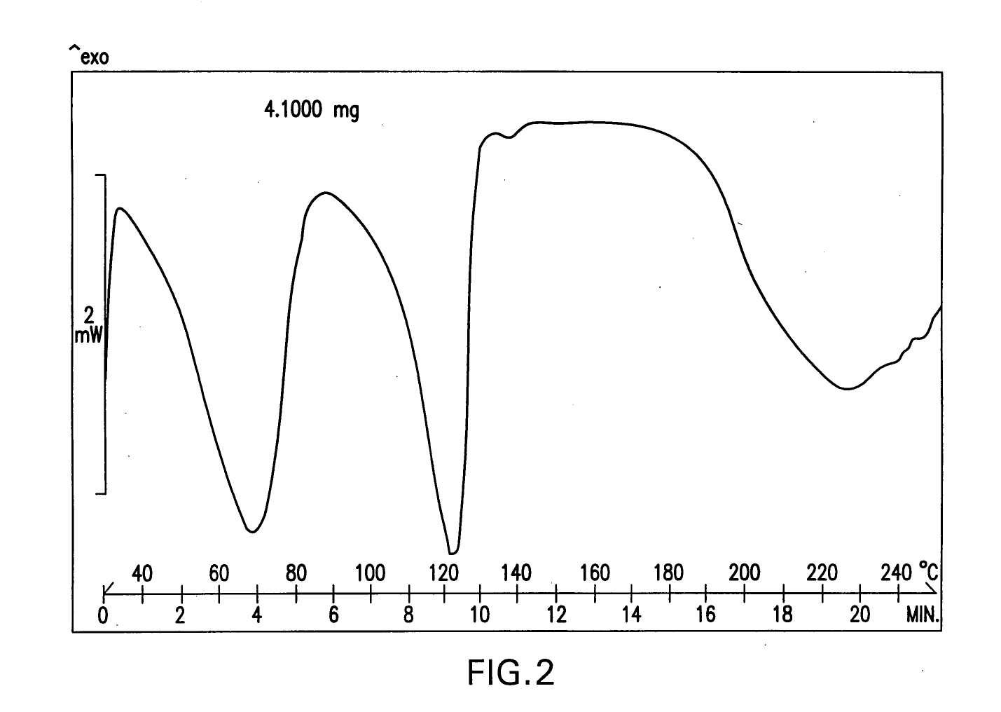

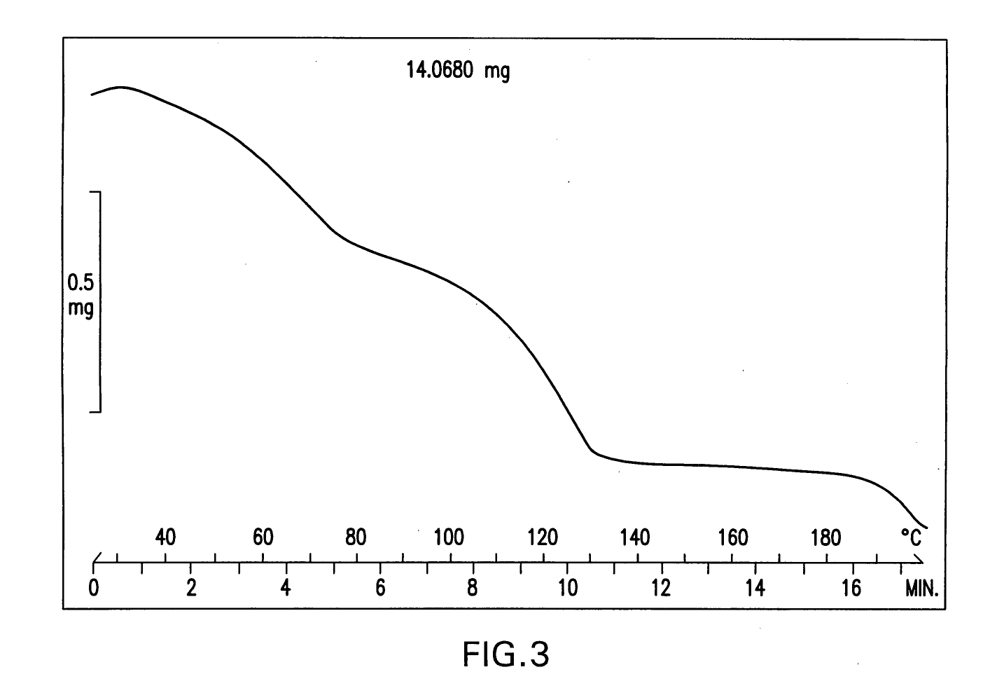

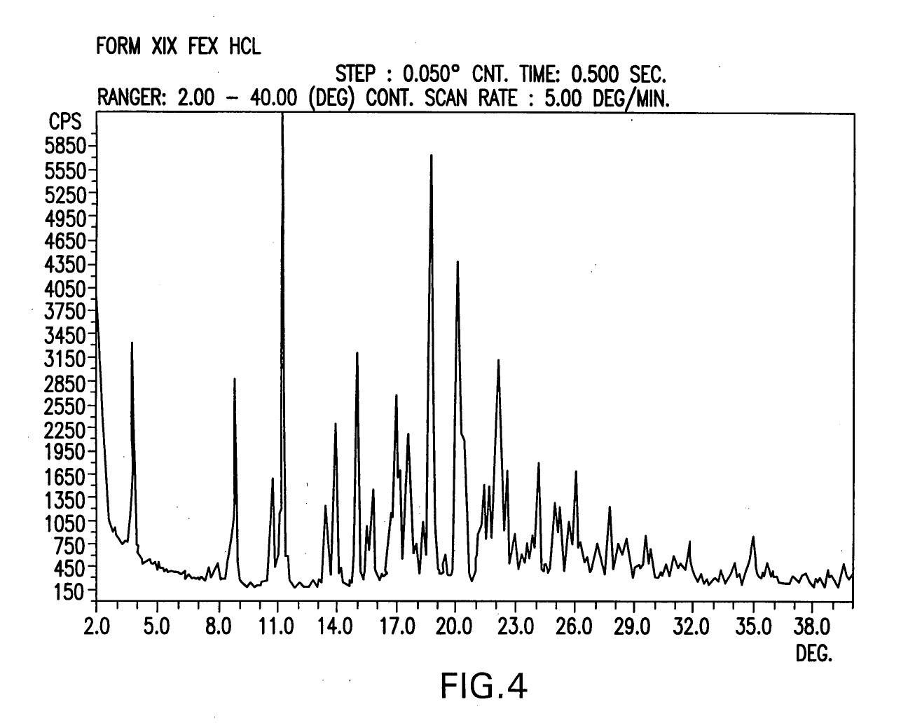

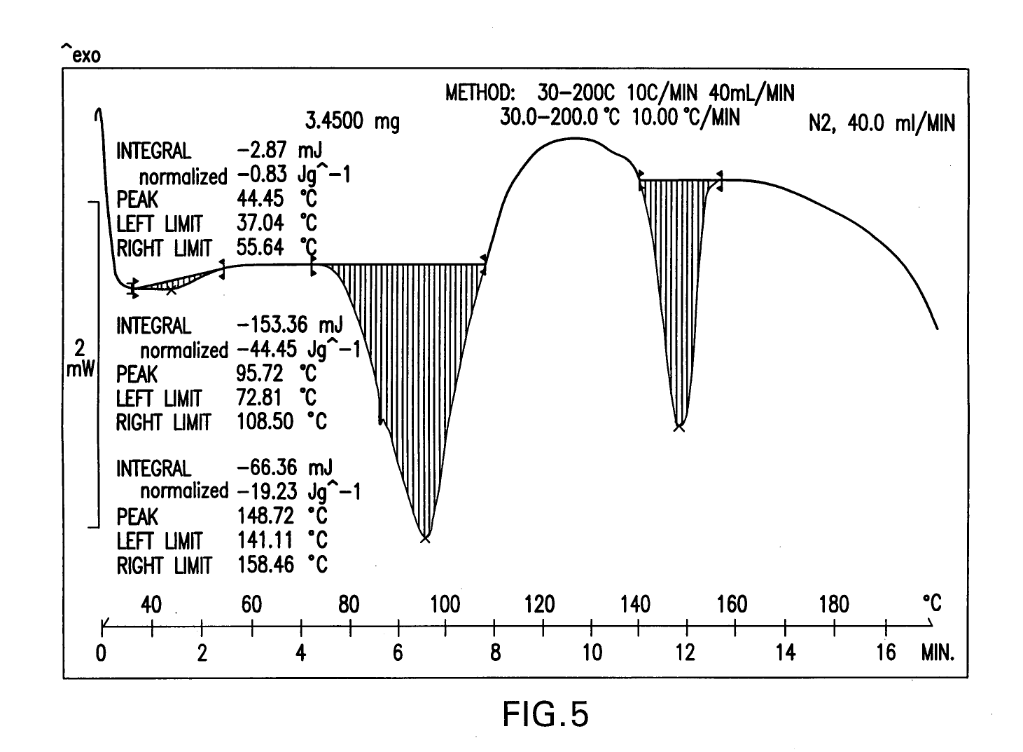

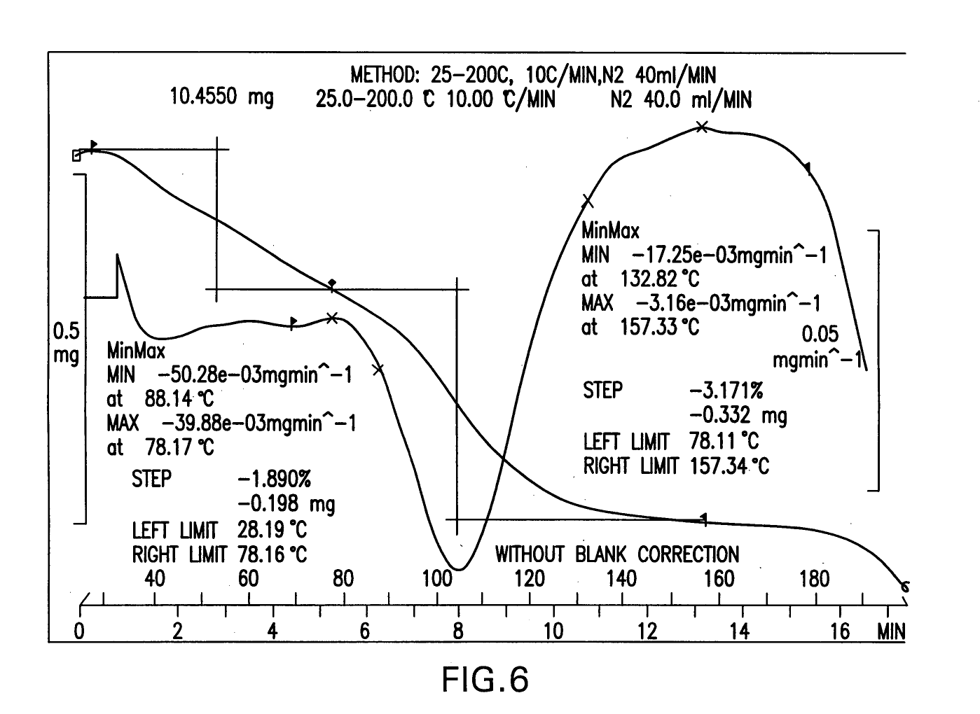

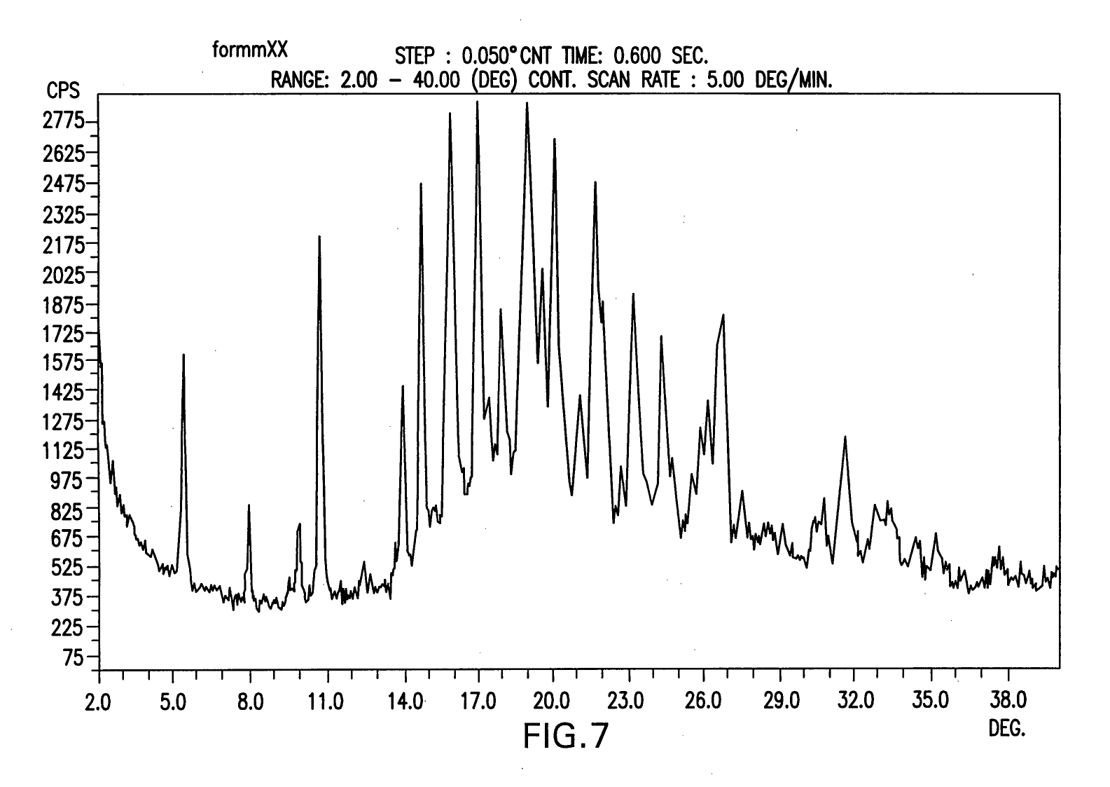

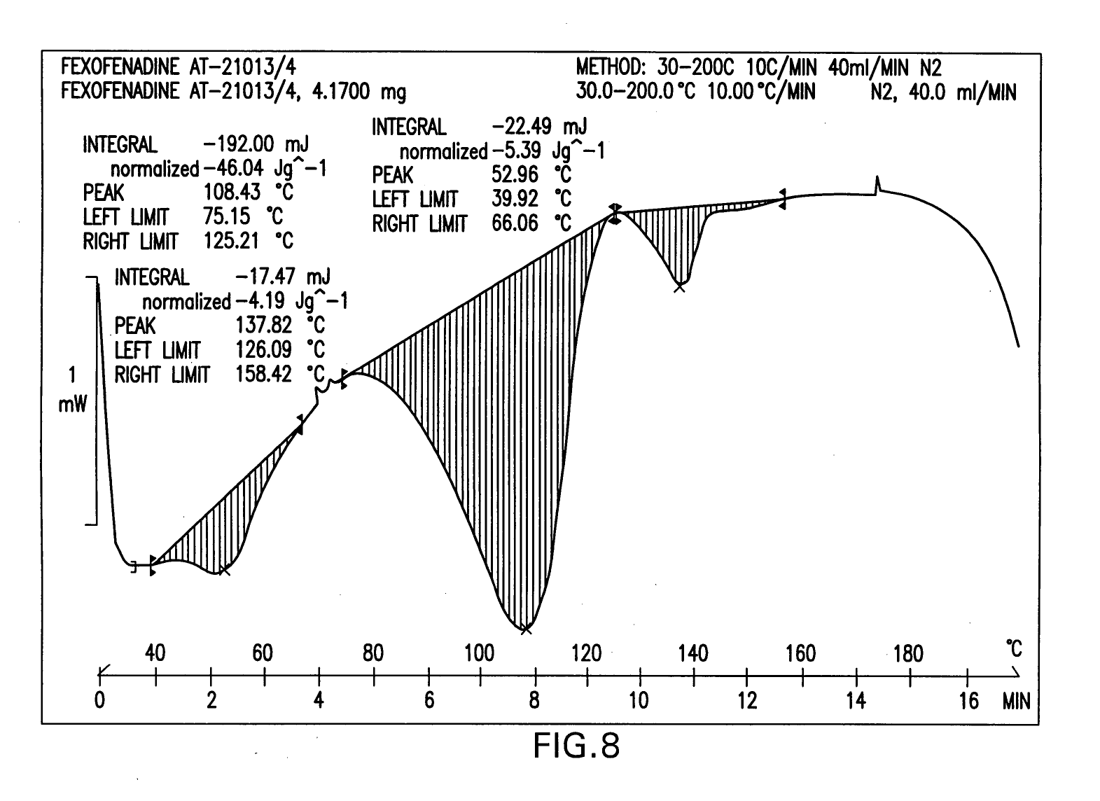

### DETAILED DESCRIPTION OF THE INVENTION

As used herein, the term “reduced pressure” refers to a pressure below one atmosphere, more preferably below about 100 mmHg, more preferably below about 50 mmHg.

As used herein, the term “vacuum” refers to a pressure below about 100 mmHg, more preferably below about 10 mmHg.

As used herein, the term “vigorous mixing” refers to mixing with an rpm of at least about 5, more preferably at least about 10, and most preferably about 20.

As used herein, the term “crystallization” refers to a process for forming crystals from a liquid or gas.

As used herein, the term “anti-solvent” refers to a liquid that when mixed with a solution of an Active Pharmaceutical Ingredient (API) reduces solubility of the API in the solution, causing crystallization or precipitation of the API, in some instances spontaneously, and in other instances with additional steps, such as seeding, cooling, scratching and/or concentrating. The API may be any polymorphic form of fexofenadine hydrochloride disclosed herein.

The polymorphic forms of the present invention are polymorphic pure, i.e., substantially free of another polymorphic form. The polymorphic pure forms of the present invention contain less than about 5%, more preferably less than about 2%, by weight of another polymorphic form of fexofenadine hydrochloride. Another polymorphic form may include any one of Form I, II, III, IV, V, VI, VIII, IX, X, XI, XII, XIII, XIV, XVI, XIX, XX and XXI of fexofenadine hydrochloride. The purity level can be determined by review of peaks in a powder XRD.

In one embodiment, the present invention provides a crystalline form of fexofenadine hydrochloride Form XIX. Form XIX has the following characteristic peaks: 3.8, 8.8, 11.3, 18.8, 20.2±0.2 deg. 2-theta. Fexofenadine hydrochloride Form XIX may be further characterized by an endothermic peak at a temperature of about 90-100° C. and an additional endotherm at about 148-155° C. in a DSC thermogram. In TGA, fexofenadine hydrochloride Form XIX shows a weight loss of about 4 to about 8% at the temperature range of 30-150° C. Appropriate powder XRD, DSC and TGA figures correspond to figure numbers 4, 5 and 6.

The present invention provides processes for preparation of fexofenadine hydrochloride Form XIX. The processes may be suitable for industrial scale. We have found that if water is added to C₁-C₄ alcohol solution in the process for preparation of Form XVI, another crystal form may be obtained, herein designated Form XIX. In order to obtain consistently Form XIX, the solvents ratio of water: C₁-C₄ alcohol, including any water present in aqueous HCl, is more than about 1.5:10 by volume. When adding smaller amount of water, the crystal form obtained depends on the filtration temperature. At a filtration temperature of less than about −5° C., more preferably less than about −10° C., and most preferably less than about −12° C., Form XVI is obtained.

Form XIX may be prepared by combining fexofenadine base with HCl and a mixture of water and C₁-C₄ alcohol in a ratio of about 1.5:10 and above to form a solution, wherein the ratio of the fexofenadine base to the alcohol is about 1:2.5 to about 1:4 (g/vol), cooling the solution, and recovering the crystalline form. Preferably cooling is carried out to a temperature of about 0° C. to about 10° C. though crystallization may be carried out at higher temperatures. Preferably, the C₁-C₄ alcohol is methanol or isopropyl alcohol. Form XIX may also be made with pre-made fexofenadine hydrochloride.

Optionally, the process may further include adding an antisolvent to the solution of fexofenadine hydrochloride. Preferably, the obtained solution is cooled before, during or after the antisolvent addition. More preferably, the antisolvent is added after cooling the solution. Crystals of fexofenadine HCl Form XIX are then recovered. Preferably, the antisolvent is a C₅ to C₁₂ saturated hydrocarbon, more preferably, cyclic or open hexane or heptane.

The present invention also provides processes for preparation of fexofenadine hydrochloride Form XVI. The processes may be suitable for industrial scale. In the present invention, Form XVI may be prepared by carrying out crystallization in the presence of low amounts of water, preferably water to C₁-C₄ alcohol ratio of about 1.2:10 and below by volume, at a sufficiently low temperature. Form XVI may be prepared by combining HCl and a mixture of water and C₁-C₄ alcohol to obtain a water to alcohol ratio of about 0.5:10 to about 1.2:10, adding fexofenadine base to the C₁-C₄ alcohol to form a solution of fexofenadine hydrochloride, cooling the solution, and recovering the crystalline Form. The ratio of water to C₁-C₄ alcohol in the solution is most preferably about 1:10 (v/v). The ratio of fexofenadine base is preferably about 1:2.5 to 1:4 (g/vol) to the alcohol. Preferably, the C₁-C₄ alcohol is methanol or isopropyl alcohol. More preferably, the C₁-C₄ alcohol is methanol. Before adding the fexofenadine base, the solution is preferably kept at a temperature of about −5° C. to about 10° C., with about 5° C. being preferred. Preferably, the fexofenadine hydrochloride solution is cooled to a temperature of at least about negative 5° C., more preferably about negative 11° C. to about negative 20° C., and most preferably about negative 12° C. To obtain a solution of the salt, the container may be agitated or filtered. It is also possible to start the process with pre-made fexofenadine hydrochloride, followed by dissolution in aqueous methanol to obtain a solution.

Optionally, the process may further comprise adding an antisolvent to the solution of fexofenadine hydrochloride. Preferably, the obtained solution is cooled before, during or after the antisolvent addition. More preferably, the antisolvent is added after cooling the solution. Crystals of fexofenadine HCl Form XVI are then recovered. Preferably, the antisolvent is a C₅ to C₁₂ saturated hydrocarbon, more preferably, cyclic or open hexane or heptane.

After cooling the solution of fexofenadine hydrochloride, the resulting heterogeneous mixture is preferably kept and stirred within the same temperature range for about 1 hour to about 1 day, most preferably of about 2 hours to about 16 hours.

In yet another embodiment of the invention, Form XVI may be prepared by exposing fexofenadine HCl Form XX to a humid atmosphere having a relative humidity of greater than about 40%, preferably about 70 to about 85%. For contacting the fexofenadine HCl Form XX to humid atmosphere, known techniques, such as a fluidized bed dryer or controlled humidity cells may be used. Preferably, for contacting the fexofenadine HCl Form XX to humid atmosphere a fluidized bed dryer is used. The time of exposure varies, and depends on the amount of material and technique used. Preferably, crystal form content of the sample is monitored by XRD. The humid atmosphere exposure process is preferably done at a temperature below about 35° C., more preferably at about room temperature. Preferably, contacting the fexofenadine HCl Form XX to humid atmosphere is performed with fluidized bed dryer at relative humidity of 70-85% RH, at a temperature of about 25° C., for about 30 minutes. Preferably, the conversion into form XVI is carried out until form XVI is at least in 80% yield.

In one embodiment, the present invention provides a crystalline Form of fexofenadine hydrochloride Form XXI. Form XXI has the following characteristic peaks: 7.2, 11.7, 14.1, 15.4, 16.9, 18.5, 23.1, and 23.9±0.2 degrees 2-theta.

Fexofenadine hydrochloride Form XXI may be prepared by combining fexofenadine base and isopropyl-alcohol with HCl to form a solution containing at least 10% water by volume relative to isopropyl alcohol, wherein ratio of fexofenadine base to isopropanol is about 1:2 and below (g/vol), cooling the solution, and recovering the crystalline form. Preferably, the solution is cooled to a temperature of about −20° C. to about 0° C., more preferably about −10° C. Cooling is preferably carried out with agitation. The process may also be carried out with pre-made fexofenadine hydrochloride.

The crystals may be recovered by conventional techniques such as filtration, decanting or centrifugation. The wet crystals obtained from the processes of the present invention may then be dried. Drying may be carried out by heating the wet crystals at ambient or reduced pressure. Preferably drying is carried out a temperature of about 50° C. to about 80° C., with about 65° C. to about 70° C. being preferred. Preferably, the pressure for drying is about below about 100 mmHg, more preferably below about 50 mmHg. Depending on the temperature or the pressure, drying may be carried out for a few days, but preferably of about 6 hours to about 24 hours, with about 16 hours being preferred.

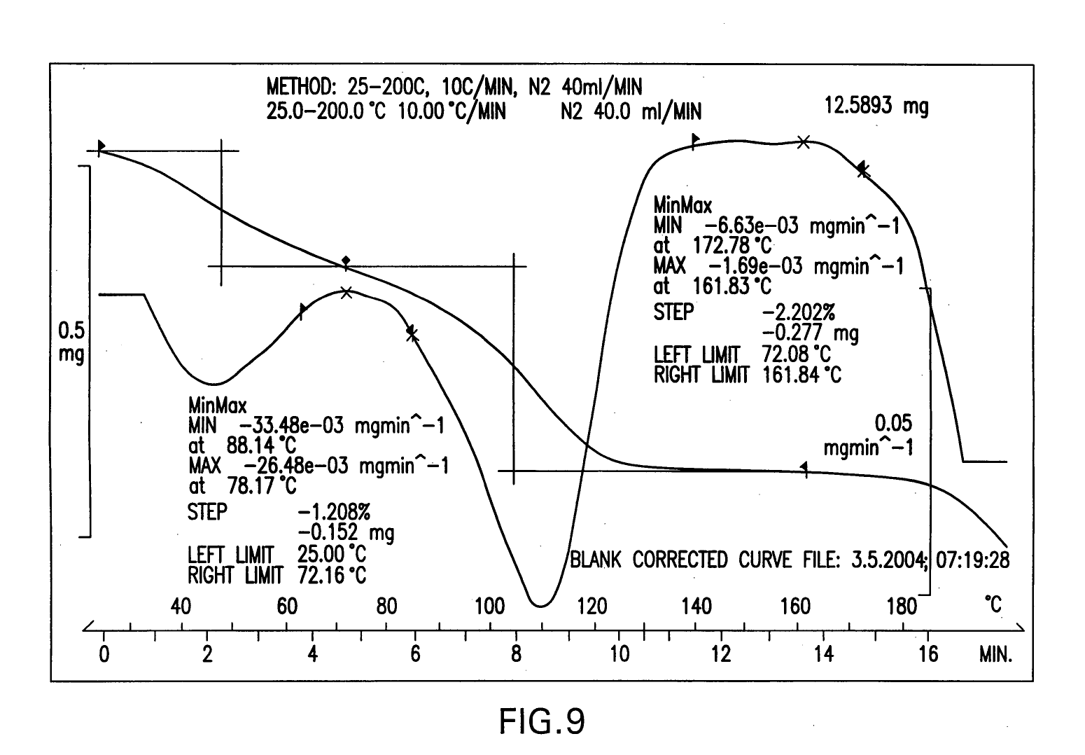

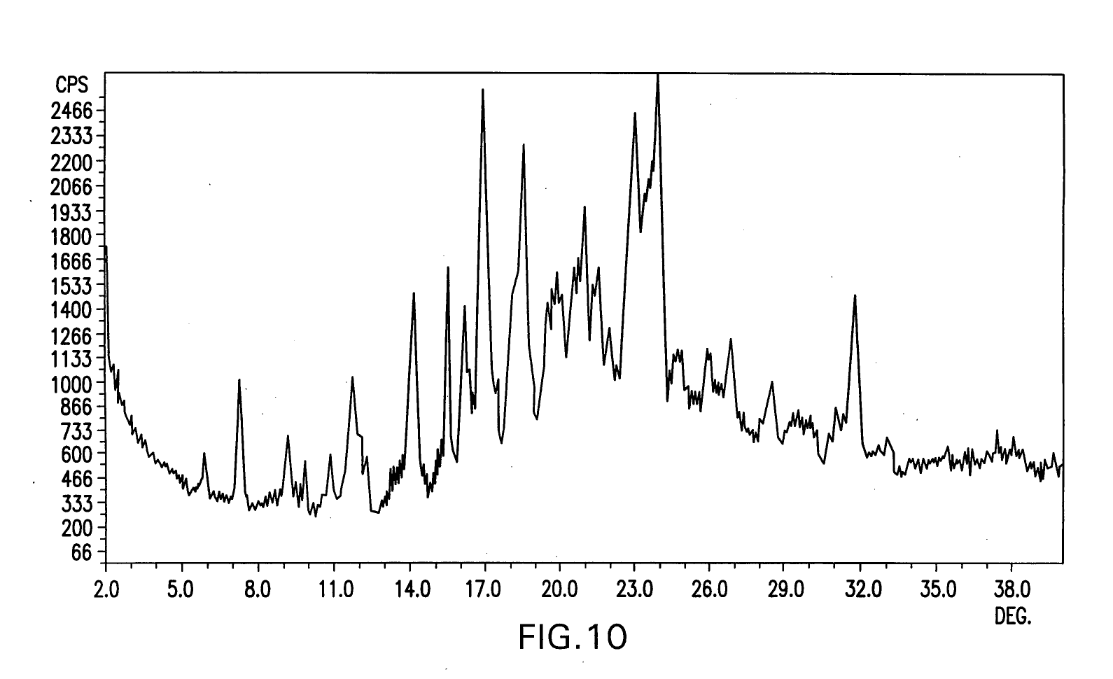

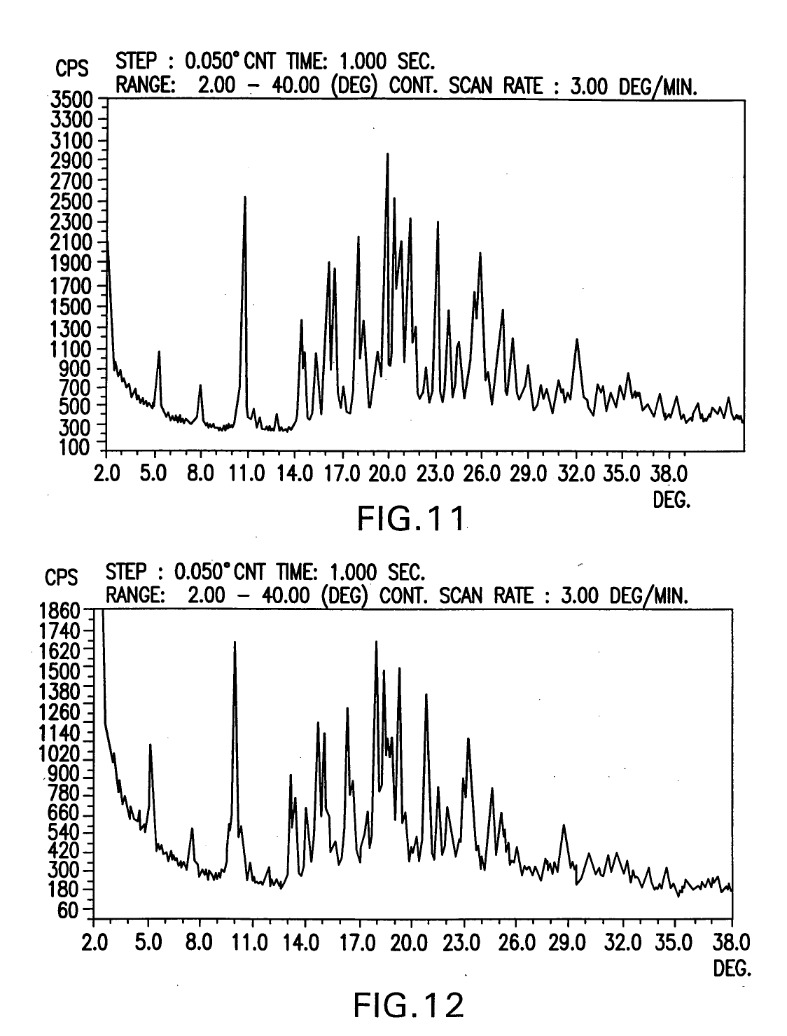

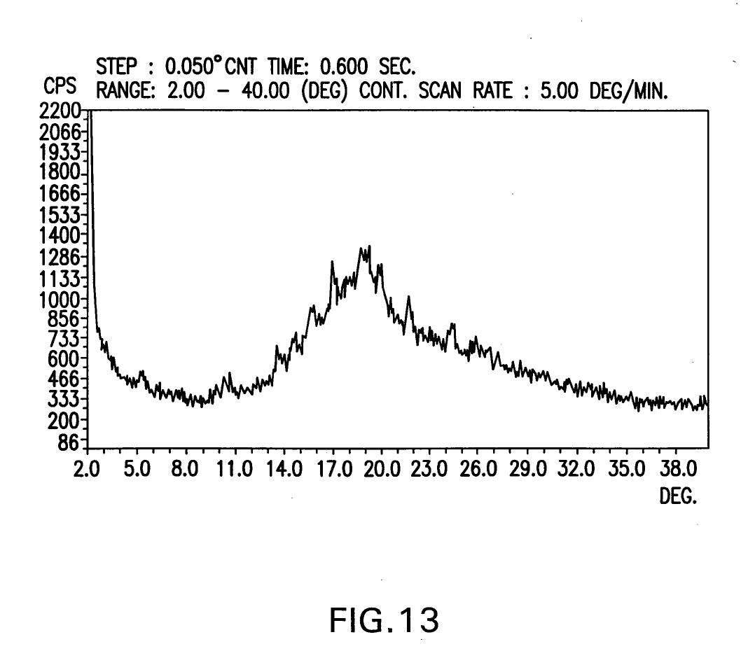

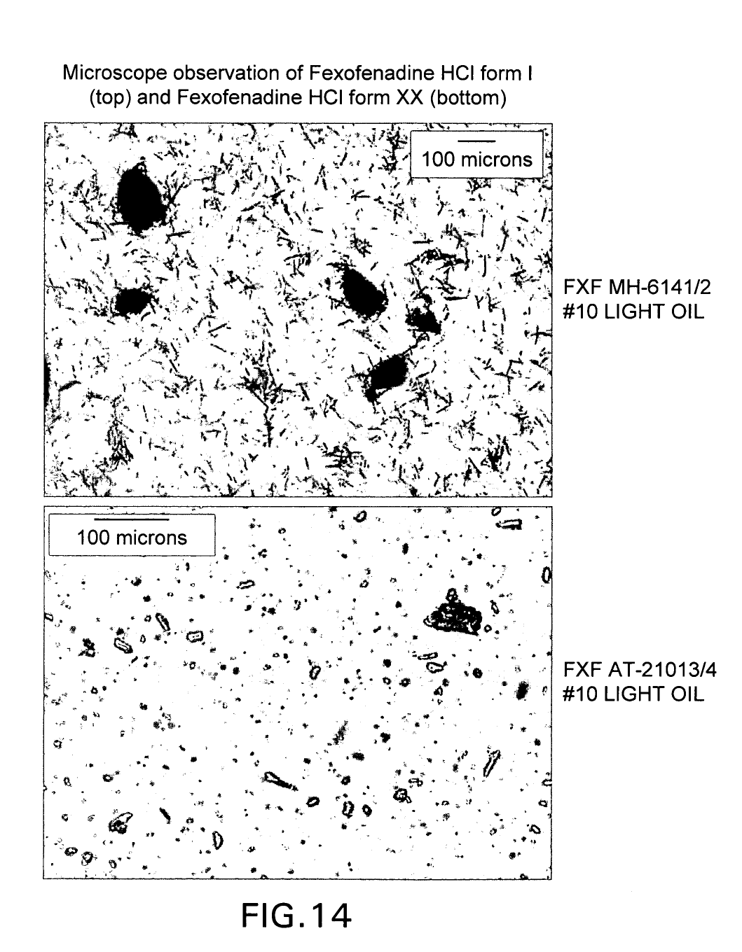

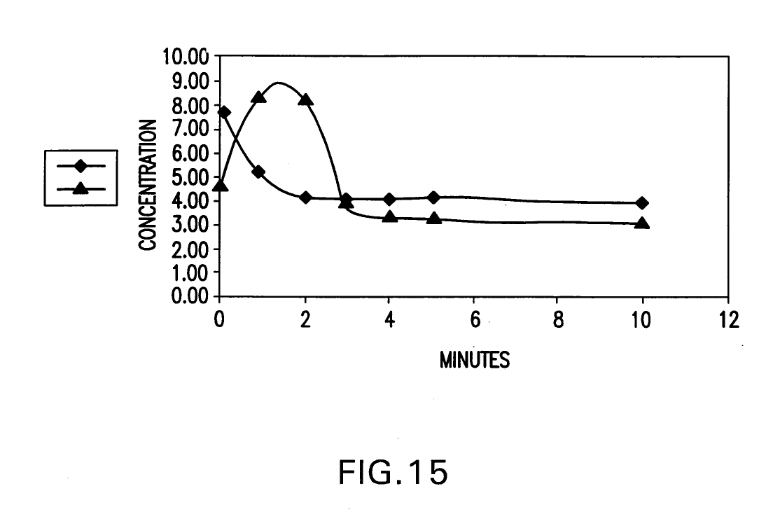

In one embodiment, the present invention provides a crystalline Form of fexofenadine hydrochloride Form XX. Form XX is characterized by a powder XRD pattern with peaks at 5.4, 10.7, 14.0, 14.7, 15.8, 17.0, 19.0, 20.0, 21.6 and 23.2±0.2 degrees 2-theta. TGA curve of fexofenadine hydrochloride Form XX (FIG. 9) shows weight loss of about 3 to about 4%. Crystalline Form XX is a hydrate. The weight loss in TGA points to a water content of about 3% to about 4% water. Appropriate PXRD, DSC and TGA figures correspond to figure numbers 7, 8 and 9.

The present invention provides processes suitable for preparation of fexofenadine hydrochloride Form XX. In the present invention, Form XX may be prepared from Form XVI by drying Form XVI under various conditions. Form XVI may be dried in the presence of seed crystals of Form XX, or dried while mixing Form XVI (before, during or after mixing). The mixing accelerates the conversion to Form XX. Fexofenadine HCl Form XX is obtained by exposing fexofenadine HCl Form XVI to low humid atmosphere of less than about 20% RH, preferably, about 0% RH. The relative amount of Form XX in relation to Form XVI increases in low humidity atmosphere, while it decreases in high humidity atmosphere. Quantity of Form XX in a mixture containing both Form XVI and Form XX may be determined by the characteristic XRD peak of Form XX at about 10.6 degrees 2θ.

In one embodiment the invention, Form XX may be prepared by drying wet fexofenadine hydrochloride Form XVI in a mixed vacuum dryer. The drying is preferably carried out at a temperature of about 60-70° C., preferably at 65° C., with vigorous mixing. The drying process is preferably monitored by XRD. Alternatively, Form XVI is dried without mixing for about two hours, followed by further drying, while mixing for about 8-32 hours, preferably for about 28 hour. The drying is preferably carried out at a temperature of about 60-70° C., more preferably at about 65° C. Preferably the mixing is vigorous.

In another embodiment the invention, Form XX may be prepared by drying wet fexofenadine hydrochloride Form XVI in a tray vacuum dryer. The drying is preferably carried out at a temperature of about 75-90° C., preferably at about 80° C. for about 15 hour. Preferably, the relative humidity should be less than 50%.

In yet another embodiment of the invention, Form XX may be prepared by drying fexofenadine hydrochloride Forms XVI and XX in a fluidized bed dryer. The drying is preferably carried out at a temperature of about 20-30° C., preferably at about 25° C. Preferably, the mixing is vigorous.

One of skill in the art would appreciate other types of dryers, such as rotary vacuum drier, spin flash drier, tunnel drier, and drum drier may also be suitable.

In another embodiment of the invention, fexofenadine hydrochloride Form XX may be prepared by feeding fexofenadine hydrochloride Form XVI into a micronizer. Form XVI may be fed either manually or by a vibratory feeder, among others. Preferably, the feed air pressure is of about 6-8 bar and the grinding air pressure is 4-7 bar. A micronizer refers to a machine that reduces the size of particles and increases surface area of particles by colliding particles with each other at high speeds.

The morphology of an active pharmaceutical substance plays an important role in drug performance, and has a profound impact on handling during milling processes and during drug product manufacturing. The known fexofenadine HCl form I contains needles morphology, while fexofenadine HCl form XX shows small rod shape particles of up to about 30 to about 40 microns. Needles shape particles are generally undesirable because they often exhibit poor flowability.

Solubility is another important property affected by solid state characteristics of the drug substance. Water solubility of form XX and form I were tested by slurry an excess amount of the samples in water, and measuring the concentration of fexofenadine HCl in the solution by HPLC. Solubility results for the known Fexofenadine HCl form I show considerable fluctuations in the concentration of Fexofenadine HCl in solution, while solubility test for Form XX shows a moderate decrease in the concentration of fexofenadine HCl in solution, until a plateau is observed. Since Solubility correlates with bioavailability, and consequently absorption and efficiency of the drug product, fluctuations in solubility are undesirable.

In another embodiment, the present invention provides a process for preparing Fexofenadine HCl amorphous. Fexofenadine HCl amorphous may be prepared by heating Fexofenadine HCl Form XVI. Heating is preferably carried out at a temperature of about 80° C. to about 100° C., preferably for at least 10 hours.

In the processes of the present invention particularly drying processes) where conversion of one polymorphic form results in another polymorphic Form, at least about a 10%, more preferably at least about a 30% and most preferably at least about a 50% conversion takes place.

Preferably, the ratio of HCl to fexofenadine base, for all the polymorphic forms above, is of about 0.9 to about 1.5, most preferably about 1 equivalent. Preferably, an about a 1:1 molar ratio or a slight excess of HCl to fexofenadine base is used.

In the processes of the present invention, the solution of HCl may be added to fexofenadine base, or vice versa. Preferably fexofenadine base is added to a container, i.e., flask or reactor, containing an aqueous solution of hydrochloric acid in alcohol.

One skilled in the art would appreciate that the polymorphs of the present invention can be selectively obtained from fexofenadine hydrochloride generally through crystallization with different recrystallization solvent systems. The starting material can be anhydrous fexofenadine hydrochloride or any fexofenadine hydrochloride hydrate or lower alcohol solvate and other solvated forms. The starting fexofenadine hydrochloride can also be in an amorphous or any crystalline crystal Form. The process can be used as a chemical purification method by using the desired form in an unacceptably pure state as starting material. The processes of the present invention can also be practiced as the last step in the methods discussed in U.S. Pat. Nos. 5,578,610, 5,589,487, 5,581,011, 5,663,412, 5,750,703, 5,994,549, 5,618,940, 5,631375, 5,644,061, 5,650,516, 5,652,370, 5,654,433, 5,663,353, 5,675,009, 5,375,693 and 6,147,216 to prepare Form XVI of fexofenadine hydrochloride.

Many processes of the present invention involve crystallization out of a solution. One skilled in the art would appreciate that the conditions concerning crystallization can be modified without affecting the Form of the polymorph obtained. For example, when mixing fexofenadine hydrochloride or free base in a solvent to Form a solution, warming of the mixture can be necessary to completely dissolve the starting material. If warming does not clarify the mixture, the mixture can be diluted or filtered. To filter, the hot mixture can be passed through paper, glass fiber or other membrane material, or a clarifying agent such as celite. Depending upon the equipment used and the concentration and temperature of the solution, the filtration apparatus may need to be preheated to avoid premature crystallization.

Pharmaceutical compositions of the present invention contain fexofenadine hydrochloride. In addition to the active ingredient(s), the pharmaceutical compositions of the present invention may contain one or more excipients. Excipients are added to the composition for a variety of purposes.

Diluents increase the bulk of a solid pharmaceutical composition and may make a pharmaceutical dosage Form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage Form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid composition and improve the accuracy of dosing. Excipients that may function as glidants include colloidal silicon dixoide, magnesium trisilicate, powdered cellulose, starch, talc and tribasic calcium phosphate.

When a dosage Form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product Form the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage Form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention hydrochloride Forms and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxy toluene, butylated hydroxyanisole and ethylenediamine tetraacetic acid may be added at levels safe for ingestion to improve storage stability.

A liquid composition according to the present invention may also contain a buffer such as guconic acid, lactic acid, citric acid or acetic acid, sodium guconate, sodium lactate, sodium citrate or sodium acetate.

Selection of excipients and the amounts to use may be readily determined by the Formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage Form and prepared by any of the methods well-known in the pharmaceutical arts.

Dosage Forms include solid dosage Forms like tablets, powders, capsules, suppositories, sachets, troches and losenges as well as liquid syrups, suspensions and elixirs.

A dosage Form of the present invention is a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be Formulated into compositions and dosage Forms according to methods known in the art.

A composition for tableting or capsule filing may be prepared by wet granulation. In wet granulation some or all of the active ingredients and excipients in powder Form are blended and then further mixed in the presence of a liquid, typically water, that causes the powders to clump up into granules. The granulate is screened and/or milled, dried and then screened and/or milled to the desired particle size. The granulate may then be tableted or other excipients may be added prior to tableting such as a glidant and or lubricant.

A tableting composition may be prepared conventionally by dry blending. For instance, the blended composition of the actives and excipients may be compacted into a slug or a sheet and then comminuted into compacted granules. The compacted granules may be compressed subsequently into a tablet.

As an alternative to dry granulation, a blended composition may be compressed directly into a compacted dosage Form using direct compression techniques. Direct compression produces a more uniform tablet without granules. Excipients that are particularly well suited to direct compression tableting include microcrystalline cellulose, spray dried lactose, dicalcium phosphate dihydrate and colloidal silica. The proper use of these and other excipients in direct compression tableting is known to those in the art with experience and skill in particular Formulation challenges of direct compression tableting.

A capsule filling of the present invention may comprise any of the aforementioned blends and granulates that were described with reference to tableting, only they are not subjected to a final tableting step.

Instruments

XRD

XRD diffraction was performed on X-Ray powder diffractometer, Scintag, variable goniometer, Cu-tube, solid state detector. A round standard aluminum sample holder with round zero background was used.

Scanning parameters: Range: 2-40 deg.2θ, Continuous Scan, Rate: 3 deg./min.

Thermal Analysis

DSC thermogram was performed on DSC821^{e}, Mettler Toledo

Sample weight: 3-5 mg

Heating rate: 10° C./min

Number of holes in the crucible: 3

TGA thermogram was performed on Mettler TG50 using standard Alumina pan.

Sample weight: 7-15 mg,

Heating rate: 10° C./min

### EXAMPLES

### Example 1

### Preparation of Fexofenadine Hydrochloride Form XVI

Methanol (120 ml), water (6 ml), and 32% HCl solution (10 g) were added to a reactor. The solution was cooled to negative 5° C. under agitation. Fexofenadine base (40 g) was added to the reactor. Agitation was continued until full dissolution was obtained. The solution was cooled under agitation to −12° C. The suspension was stirred for 2 to 16 hours at −12° C. The product was filtered. Pure fexofenadine HCl Form XVI was obtained. The resulting wet cake of fexofenadine HCl Form XVI was dried under vacuum (10 mmHg) at a temperature of 65° C. to 80° C. After 16 hours of drying, pure fexofenadine Form XVI was obtained.

### Example 2

### Preparation of Fexofenadine Hydrochloride Form XIX

6 g of HCl 32% (leq) and 2 vol. of methanol were introduced into a 1 liter reactor. 25 gr of Fexofenadine Base pure were dissolved at 25° C. under stirring 20 vol. of Heptane and 2 vol. of soft water were added. The crystals obtained were filtered at 20° C. Fexofenadine HCl Form XIX was obtained.

### Example 3

### Preparation of Fexofenadine Hydrochloride Form XIX

2 vol. of methanol and 1 vol. of soft water were Introduce into a 1 liter reactor. 25 g of Fexofenadine Base pure were dissolved at 25° C. under stirring. HCl 32% (1 eq.) was added. The mixture was heated to 40° C. into complete dissolution, and than cooled to 20° C. The mixture was stirred for 30 min. 2 vol of Heptane were then added at 25° C. The mixture was cooled to 15° C. within 1 hr, and then filtered. Fexofenadine HCl Form XIX was obtained.

### Example 4

### Preparation of Fexofenadine Hydrochloride Form XIX

Methanol (120 ml), water (12 ml) and 32% HCl solution (10 g) were added to a reactor. The solution was cooled down to 5° C. under agitation. Fexofenadine base (40 g) was added to the reactor. Agitation was continued until full dissolution was obtained. The solution was cooled down (under agitation) to (−12)° C. After stirring the suspension for additional 2-16 hours, the product was filtered and dried under vacuum in a temperature of 65-80° C. Fexofenadine HCl Form XIX was obtained immediately after filtration as a wet product and also after drying. Fexofenadine HCl Form XIX was obtained. Water content by KF=7.1%

### Example 5

### Preparation of Fexofenadine Hydrochloride Form XIX

Methanol (80 ml) and water (8 ml) and 32% HCl solution (10 g) were added to a reactor. The solution was cooled down to 5° C. under agitation. Fexofenadine base (40 g) was added to the reactor. Agitation continued until full dissolution was obtained. The solution was cooled down under agitation to −12° C. After stirring the suspension for an additional 2-16 hours, the product was filtered and dried under vacuum in a temperature of 65-80° C. for 16 h. Fexofenadine HCl Form XIX was obtained immediately after filtration as a wet product and also after drying. Water content by KF=4.4%.

### Example 6

### Preparation of Fexofenadine Hydrochloride Form XIX, Mixed with Form XVI

Methanol (120 ml), water (6 ml) and 32% HCl solution (10 g) were added to a reactor. The solution was cooled down to 5° C. under agitation. Fexofenadine base (40 g) was added to the reactor. Agitation was continued until full dissolution was obtained. The solution was cooled down (under agitation). Samples were taken out at 0° C., −5° C. and −10° C. The mixture was cooled down to −12° C. After stirring the suspension for additional 2-16 hours, the product was filtered, and dried under vacuum at a temperature of 65-80° C. for 6 h.

The following table describes the crystal Form obtained during the cooling stage progress:

| | Filtration temperature |
|---|---|
| Crystal Form | T(° C.) |
| XVI + XIX | 0 |
| XVI | −5 |
| XVI | −10 |
| XVI | −12 |

### Example 7

### Preparation of Fexofenadine Hydrochloride Form XVI

Isopropyl-alcohol (150 ml) and 32% HCl solution (11.5 g) were added to a reactor. The solution was cooled down to 10° C. under agitation. Fexofenadine base (50 g) was added to a reactor. Agitation continued until full dissolution was obtained. The solution was cooled down (under agitation) to −12° C. Heptane (5 ml) was added to the reactor and cloudiness appeared. After stirring the suspension for additional 2-16 hours, the product was filtered. Pure Fexofenadine HCl Form XVI was obtained. The wet cake was dried under vacuum at a temperature of 65-80° C. After 16 hrs of drying fexofenadine, Form XVI was obtained.

### Example 8

### Preparation of Fexofenadine HCl Form XX

Fexofenadine HCl Form XVI (10 g) as a wet sample was added to a mixed vacuum dryer. The sample was dried at a temperature of 65° C. under vacuum and a vigorous mixing (˜20 rpm) over a period of 12 hours. After 12 hours, fexofenadine HCl Form XX was obtained.

### Example 9

### Preparation of Fexofenadine HCl Form XX, Mixed with Form XVI

Fexofenadine HCl Form XVI (400 g.) as wet sample was added to a mixed vacuum dryer. The material was dried in a temperature of 65° C. under vacuum. In the first two hours the material was dried without any mixing (static drying). After two hours, the material was vigorously mixed (˜20 rpm). The following table describes the change in polymorphic Form during the drying stage progress:

| Time (hours) | Form | Remarks |
|---|---|---|
| 2 | XVI | Static drying |
| 4 | XVI | Mixed drying |
| 8 | XVI + XX | Mixed drying |
| 10 | XVI + XX | Mixed drying |
| 12 | XVI + XX | Mixed drying |
| 14 | XVI + XX | Mixed drying |
| 18 | XVI + XX | Mixed drying |
| 22 | XVI + XX | Mixed drying |
| 28 | XX | Mixed drying |
| 32 | XX | Mixed drying |

### Example 10

### Preparation of Form XX

Fexofenadine HCl Form XVI (40 g) was added to a tray vacuum dryer. The sample was dried in a temperature of 80° C. under vacuum. After 15 hours of drying, fexofenadine HCl Form XX was obtained.

### Example 11

### Preparation of Form XX

Fexofenadine HCl (40 g) containing a mixture of Form XVI and Form XX was added to a fluidized bed dryer (with a flow of dry nitrogen). The material was dried in a temperature of 25° C. The following table describes the change in polymorphic Form progress:

| Time (hours) | Form |
|---|---|
| 4 | XX |
| 8 | XX |
| 12 | XX |

### Example 12

### Preparation of Form XX

A laboratory micronizer, (model: Sturtevant qualification micronizer 50 mm or Atritor 50 mm) was used. Fexofenadine HCl Form XVI was fed into the micronizer by a vibratory feeder. The feed air pressure was between 6-8 bar and the grinding air pressure 4-7 bar. Fexofenadine HCl Form XX was obtained

### Example 13

### Preparation of Form XXI

Isopropanol (80 ml), water (2 ml), and 32% HCl solution (10 g) were added to a reactor. The solution was cooled to 5° C. under agitation. Fexofenadine base (40 g) was added to the reactor. Agitation continued until full dissolution was obtained. The solution was cooled down under agitation to −10° C. After stirring the suspension for an additional 1 hour, the product was filtered and dried under vacuum in a temperature of 65° C. for 16 h. Fexofenadine HCl Form XIX (27 g) was obtained.

### Example 14

### Preparation of Fexofenadine Form XVI

38 Kg Fexofenadine HCl Form XX was fluidized in Fluidized Bed Dryer at 25° C. and relative humidity of 70-85% for 30 minutes. 39 Kg Fexofenadine HCl Form XVI were obtained.

### Example 15

### Preparation of Fexofenadine Form XVI

A reactor was charged with 73.8 kg methanolic solution (5% HCl in methanol), 8 liter of methanol, and 5 kg of process water. The reactor was cooled to a temp below 5° C., and Fexofenadine Base Pure (50 kg) was gradually added, while the temp in the reactor is kept below 5° C. The solution was filtered from foreign particles, and then seeding was performed. After the material start to precipitate, the reactor content is cooled to below (−15° C.). The reactor content was filtered and the filter cake was washed with 300 liter of Heptane. The material was dried in a vacuum dryer at 60-70° C. and then it was dried at Fluidized Bed Dryer at 60-70° C. The material was milled and then it was fluidized in Fluidized Bed Dryer at 25° C. and relative humidity of 70-85% for 30minutes. Fexofenadine HCl Form XVI was obtained. Water content by KF is 8%.

### Example 16

### Preparation of Fexofenadine HCl Form XVI, Mixed with Form XX

200 mg of Fexofenadine HCl Form XX was spread as a thin layer on an open dish, and put in controlled humidity cells of 40, 60 and 80% RH for 9 days at 30C, and then tested by XRD:

| | | Crystal Form |
|---|---|---|
| % RH | Water content | content by XRD |
| 40 | 4.9 | XVI + XX |
| 60 | 5.2 | XVI + XX |
| 80 | 7.8 | XVI |

### Example 17

### Preparation of Fexofenadine HCl Form XVI, Mixed with Form XX

200 mg of Fexofenadine HCl Form XVI was spread as a thin layer on an open dish, and then put in controlled humidity cells of 0, 20, 40, 60 and 80% RH for 7 days at room temperature. The samples were tested by XRD and by KF titration.

| | | Crystal Form |
|---|---|---|
| % RH | Water content | content by XRD |
| 0 | 4.2 | XVI + XX |
| 20 | 7.0 | XVI |
| 40 | 7.3 | XVI |
| 60 | 7.3 | XVI |
| 80 | 8.3 | XVI |

### Example 18

### Preparation of Fexofenadine HCl Form XVI, Mixed with Form XX

200 mg of a sample containing a mixture of Fexofenadine HCl Form XVI and Fexofenadine HCl Form XX was spread as a thin layer on an open dish, and than put in controlled humidity cells of 0, 20, 40, 60 and 80% RH for 7 days at room temperature, and than tested by XRD and by KF titration.

| | | Crystal Form content |
|---|---|---|
| % RH | Water content | by XRD |
| 0 | 4.2 | 85% XVI, 15% Form XX |
| 20 | 7.0 | 93% XVI, 7% Form XX |
| 40 | 7.3 | 96% XVI, 4% Form XX |
| 60 | 7.3 | 96% XVI, 4% Form XX |
| 80 | 8.3 | XVI |

### Example 19

### Preparation of Fexofenadine HCl Form XVI, Mixed with Amorphous Form

0.5 g of Fexofenadine HCl Form XVI was heated under atmospheric pressure at 60, 80 and 100° C. The samples were than tested by XRD.

| Initial Form | Temp. | time | Resulted Form |
|---|---|---|---|
| XVI | 65 C. | 10 h | XVI |
| XVI | 80 C. | 10 h | XVI + Amorphous |
| XVI | 100 C. | 10 h | XVI + Amorphous |

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used for guidance. All references mentioned herein are incorporated in their entirety

1. A crystalline form of fexofenadine HCl (Form XIX) characterized by a powder XRD pattern with peaks at 3.8, 8.8, 11.3, 18.8, 20.2±0.2 deg. 2θ.

2. The crystalline form of claim 1, wherein the crystalline form is further characterized by at least one of:
a) a DSC profile having a first endothermic peak at a temperature of about 90° C. to about 100° C. and a second endotherm at a temperature of about 148 to about 155° C.; or
b) a weight loss of about 4 to about 8% at temperature range of 30° C. to 150° C. by TGA.

3. A powder of crystalline form of claim 1 comprising less than 5% by weight of any other crystalline form of fexofenadine hydrochloride.

4. A process for preparing crystalline fexofenadine hydrochloride of claim 1 comprising:
a) preparing a solution of fexofenadine hydrochloride in C₁-C₄ alcohol having at least about 15% water by volume relative to the C₁-C₄ alcohol, wherein ratio of fexofenadine base used to prepare the fexofenadine hydrochloride to the C₁-C₄ alcohol is about 1:2.5 to about 1:4 (g/vol);
b) cooling the solution to crystallize the crystalline form; and
c) recovering the crystalline form.

5. The process of claim 4, wherein cooling is carried out to a temperature of about 0° C. to about 10° C.

6. The process of claim 4, wherein the C₁-C₄ alcohol is methanol.

7. The process of claim 4, wherein prior to the recovering step an anti-solvent is added to the solution.

8. The process of claim 7, wherein the anti-solvent is a C₅ to C₁₂ saturated hydrocarbon.

9. The process of claim 4, wherein the crystalline fexofenadine hydrochloride recovered has less than about 5% by weight of any other crystalline form of fexofenadine hydrochloride.

10. The process of claim 9, wherein any other crystalline forms are present in less than about 2% by weight.

11. A process for preparing crystalline fexofenadine hydrochloride (Form XVI) with peaks at 10.1, 15.2, 18.6, 19.2, 20.1 ±0.2 degrees two theta comprising:
a) preparing a solution of fexofenadine hydrochloride in a mixture of water and C₁-C₄ alcohol having less than about 12% water by volume relative to the alcohol;
b) cooling the solution to crystallize the crystalline form; and
c) recovering the crystalline form.

12. The process of claim 11, wherein the C₁-C₄ alcohol is selected from the group consisting of methanol and isopropyl alcohol.

13. The process of claim 11, wherein amount of water is of about 5% to about 12% by volume.

14. The process of claim 13, wherein amount of water is about 10%.

15. The process of claim 11, wherein the solution is cooled to a temperature of less than about negative 5° C.

16. The process of claim 15, wherein the solution is cooled to a temperature of less than about negative 12° C.

17. The process of claim 11, wherein prior to recovering the crystalline form an anti-solvent is added to the solution.

18. The process of claim 17, wherein the anti-solvent is a C₅ to C₁₂ saturated hydrocarbon.

19. The process of claim 11, wherein the crystalline fexofenadine hydrochloride recovered has less than about 5% by weight of any other crystalline form of fexofenadine hydrochloride.

20. The process of claim 19, wherein any other crystalline forms are present in less than about 2% by weight.

21. A crystalline Form of fexofenadine HCl (Form XXI) characterized by a powder XRD pattern with peaks at 7.2, 11.7, 14.1, 15.4, 16.9, 18.5, 23.1, and 23.9±0.2 deg. 2θ.

22. A powder of crystalline form of claim 21 comprising less than 5% by weight another crystalline form fexofenadine hydrochloride.

23. A process for preparing the crystalline fexofenadine hydrochloride of claim 21 comprising:
a) preparing a solution of fexofenadine HCl in isopropanol having at least about 10% water by volume, wherein the ratio of fexofenadine base used to prepare the fexofenadine hydrochloride to isopropanol is no more than about 1:2 (g/vol);
b) cooling the solution to crystallize the crystalline form; and
c) recovering the crystalline form.

24. The process of claim 23, wherein the solution is cooled to a temperature of about −20° C. to about 0° C.

25. The process of claim 24, wherein the solution is cooled to a temperature of about −10° C.

26. A crystalline form of Fexofenadine HCl (Form XX) characterized by the XRD peaks at: 5.4, 10.7, 14.0, 14.7, 15.8, 17.0, 19.0, 20.0, 21.6 and 23.2±0.2 deg. 2θ.

27. The crystalline form of claim 26 further characterized by a DSC profile with a first endothermic peak at a temperature of about 50-55° C. and a second endotherm at a temperature of about 100° C. and about 140° C.

28. A powder of crystalline form of claim 26 comprising less than 5% by weight another crystalline form of fexofenadine hydrochloride.

29. A process for preparing crystalline fexofenadine hydrochloride of claim 26 comprising drying for a sufficient time a crystalline fexofenadine hydrochloride (Form XVI) having a powder XRD pattern with peaks at 10.1, 15.2, 18.6, 19.2, 20.1±0.2.

30. The process of claim 29, wherein drying is carried out for at least about 10 hours.

31. The process of claim 29, wherein the drying is carried out with one of
a) a tray dryer;
b) mixed vacuum dryer; or
c) fluidized bed drier.

32. The process of claim 31, wherein the tray dryer is a tray vacuum dryer.

33. The process of claim 31, wherein the drying is carried out at a temperature of about 75° C. to 90° C.

34. The process of claim 31, wherein the mixed vacuum drying is carried out at a temperature of about 60° C. to about 70° C.

35. The process of claim 31, wherein the drying with the fluidized bed drier carried out at a temperature of about 20° C. to about 30° C.

36. The process of claim 29, wherein the fexofenadine hydrochloride is vigorously mixed during drying.

37. The process of claim 29, wherein the fexofenadine hydrochloride is seeded during or after drying.

38. The process of claim 29, wherein drying is carried out under vacuum.

39. A process for preparing crystalline fexofenadine hydrochloride of claim 27 comprising micronizing fexofenadine hydrochloride Form XVI with a micronizer.

40. The process of claim 39, wherein feed air pressure into the micronizer is of about 6 to about 8 bar.

41. The process of claim 39, wherein grinding air pressure of the micronizer is of about 4 to about 7 bar.

42. A process for converting crystalline fexofenadine hydrochloride Form XX to crystalline fexofenadine HCl (Form XVI) with peaks at 10.1, 15.2, 18.6, 19.2, 20.1±0.2 comprising exposing Fexofenadine HCl Form XX to a relative humidity of greater than about 40%.

43. The process of claim 42, wherein the relative humidity is about 70% to about 85%.

44. The process of claim 42, wherein fluidized bed or controlled humidity cells are used.

45. The process of claim 42, wherein the crystalline form is obtained with at least 80% yield.

46. The process of claim 42, wherein the temperatures is below about 35° C.

47. The process of claim 46, wherein the temperatures is about room temperature.

48. A process for preparing fexofenadine HCl amorphous comprising heating crystalline fexofenadine hydrochloride (Form XVI) with peaks at 10.1, 15.2, 18.6, 19.2, 20.1±0.2.

49. The process of claim 48, wherein the temperature is about 80° C. to about 100° C.

50. A pharmaceutical composition comprising a crystalline fexofenadine hydrochloride selected from the group consisting of Form XIX, XX, XXI and mixtures thereof, and a pharmaceutically acceptable excipient.

51. A pharmaceutical composition comprising a crystalline fexofenadine hydrochloride selected from the group consisting of Form XIX, XX, XXI and mixtures thereof, and a pharmaceutically acceptable excipient, for use in reducing serotonin re-uptake in a mammal in need thereof.
